(19) United States
(12) Patent Application Publication
(10) Pub. No.: US 2009/0311791 A1
(43) Pub. Date: Dec. 17, 2009

(54) ALPHA-HEMOLYSIN DELETION MUTATION OF SES-PRODUCING STAPHYLOCOCCUS AUREUS AND CONSTRUCT THEREOF

(76) Inventors: Chen Juyu, Shenyang (CN); Chen Yan, Shenyang (CN); Zhang Huiwen, Shenyang (CN); Wang Xiaogang, Shenyang (CN); Xu Mingkai, Shenyang (CN)

Correspondence Address: LEONG C LEI, PMB # 1008, 1867 YGNACIO VALLEY ROAD, WALNUT CREEK, CA, 94598, US

(21) Appl. No.: 12/140,291

(22) Filed: Jun. 17, 2008

(51) Int. Cl. C12N 15/74; C12N 1/21

(52) U.S. Cl. 435/471; 435/252.3

(57) ABSTRACT

The present invention relates to an α-hemolysin-deletion mutant of SEs-producing Staphylococcus aureus and a construction method thereof. Said mutant is a genetically engineered strain obtained by deleting an α-hemolysin (α-HL) gene of a wild type Staphylococcus aureus strain by a homologous recombination method. The genetically engineered strain has the same genetic background as the wild strain except no α-hemolysin is produced.

### FIELD OF INVENTION

The present invention relates in general to gene engineering and biotechnologies. More specifically, the invention provides a new α-hemolysin-deletion mutant of Staphylococcus aureus for staphylococcal enterotoxins (SEs) producing.

### BACKGROUND OF THE INVENTION

(a) Technical Field of the Invention

The present invention falls in the bio-technical field, more specifically, is related to the α-hemolysin-deletion mutant of SEs-producing Staphylococcus aureus and construction methods thereof.

(b) Description of the Prior Art

Gene knockout is a molecular biological technique developed in 1980s, which involves direct inactivation or deletion of a specific gene by a genetic engineering method. With flourishing development of molecular biology and post-genomic techniques, gene knockout techniques have reached an astonishing level. Through such gene knockout techniques, one is able to not only directly reconstruct the specific genes, but also understand the structures and functions of unknown genes. Owing to these advantages, gene knockout has been widely used in construction of genetic engineered strains of microorganisms.

Hemolysin (HL) is an exotoxin produced by Staphylococcus aureus during its growth. Hemolysin includes four subtypes: α, β, γ and δ. α-Hemolysin has strong hemolytic effect on red blood cells of mammalian animals, including human beings, and thus is considered as one of the major pathogenic factors of the diseases caused by Staphylococcus aureus. Staphylococcal superantigens can specifically stimulate a large number of T cells bearing particular Vβ elements in their T-cell receptor beta chain (McCormick et al., Annu. Rev. Microbiol. 55: 77-104, 2001). These enterotoxins-containing anti-tumor products obtained from fermentation of Staphylococcus aureus also contains α-hemolysin and hence may cause toxic side effects when used in human beings. This limits their use in medical field. Therefore, it is desired to develop a method for constructing an α-hemolysin-deletion mutant of SEs-producing Staphylococcus aureus, so that its enterotoxins-containing anti-tumor product may have wider medical use.

### SUMMARY OF THE INVENTION

The primary purpose of the present invention is to provide an α-hemolysin-deletion mutant of SEs-producing Staphylococcus aureus.

The present invention has another object to provide a method for constructing the α-hemolysin-deletion mutant of SEs-producing Staphylococcus aureus by homologous recombination, comprising the following steps: constructing a corresponding vector wherein α-hemolysin gene has been knocked out; transforming a wild type SEs-producing Staphylococcus aureus strain with said vector; and selecting an α-hemolysin-deletion mutant of SEs-producing Staphylococcus aureus from the transformed strains. The genetically engineered α-hemolysin-deletion mutant thus obtained produces α-hemolysin free, but still produces enterotoxins of anti-tumor products.

The foregoing object and summary provide only a brief introduction to the present invention. To fully appreciate these and other objects of the present invention as well as the invention itself all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 shows the result of agar gel electrophoresis for PCR amplification of Hla-u, Hla-d, Neor and the gene-deleted fragment Hu-Neor-Hd, wherein 1 represents DL-2000 as DNA molecular weight standard; 2 to 5 respectively represent amplification products of gene fragments Hla-u, Hla-d, Neor and Hu-Neor-Hd; and 6 represents 200 bp DNA ladder.

FIG. 2 shows the result of agar gel electrophoresis for restriction enzyme digestion of the gene-deleted vector pMHL-α and PCR amplification of Hu-Neor-Hd, wherein 1 represents λ-HindIII molecular weight marker, 2 represents BamHI digestion product of pMHL-α, 3 represents PCR amplification product of Hu-Neor-Hd and 4 represents 200 bp DNA ladder.

FIG. 3 shows the result of agar gel electrophoresis for PCR amplification of SEC2 gene, wherein 1 represents DL-2000 as DNA molecular weight standard; 2 represents the PCR products of SEC2 gene by using the genomic DNA of α-hemolysin-deletion mutant of SEs-producing Staphylococcus aureus as template, 3 represents the PCR products of SEC2 gene by using the genomic DNA of Staphylococcus aureus CGMCC0165 before mutation as template. It can be seen from the figure that SEC2 gene before and after α-hemolysin gene knockout had no difference.

FIG. 4 shows the result of agar gel electrophoresis for PCR amplification products using the genomic DNA of the wild type strain CGMCC0165 and the genomic DNA of α-HL gene-deleted strain as template, wherein M represents DL-2000 as DNA molecular weight standard; 1 and 2 represent the PCR amplification products using the genomic DNA of Staphylococcus aureus CGMCC0165 as template, and 3 and 4 represent the PCR amplification products using the genomic DNA of α-HL gene-deleted strain as template. It can be seen from the figure that a 1686 bp PCR product was obtained by using the genomic DNA of α-hemolysin-deletion mutant of Staphylococcus aureus as template and a 1497 bp PCR product was obtained by using the genomic DNA of Staphylococcus aureus CGMCC0165 as template. This proves that α-hemolysin gene in Staphylococcus aureus CGMCC0165 was successfully replaced by Neor gene and α-hemolysin-deletion mutant of Staphylococcus aureus was successfully constructed.

FIG. 5 shows the result of hemolytic analysis of the α-hemolysin-deletion mutant of SEs-producing Staphylococcus aureus. In the figure, No. 1: no significant hemolysis circle was observed for the α-hemolysin-deletion mutant of SEs-producing Staphylococcus aureus after incubation on rabbit blood agar plate for 2 to 4 days; No. 2: significant hemolysis circle was observed for the wild type SEs-producing Staphylococcus aureus strain CGMCC0165 and the hemolysis circle was enlarged with time. This indicates that the method for constructing the α-hemolysin-deletion mutant of SEs-producing Staphylococcus aureus according to the present invention is feasible, and the α-hemolysin-deletion mutant of SEs-producing Staphylococcus aureus obtained by this method is very useful in production of the enterotoxins-containing anti-tumor drugs with much less side effects so that the enterotoxins-containing anti-tumor drugs can have use in clinical anti-tumor therapy.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

In order to achieve the above objects, the following strategies are adopted. The α-hemolysin-deletion mutant of SEs-producing Staphylococcus aureus is a genetically engineered strain obtained by deleting α-hemolysin gene from a wild type SEs-producing Staphylococcus aureus strain via a homologous recombination method.

The wild type SEs-producing Staphylococcus aureus strain has been deposited in China General Microbiological Culture Collection Center under accession no. CGMCC0165.

The α-hemolysin-deletion mutant is constructed by the following steps:

1.) amplifying the gene fragment Hla-u which is homologous to the upstream of α-HL gene, the gene fragment Hla-d which is homologous to the downstream of α-HL gene, and neomycin resistance (Neor) gene for substituting native α-HL gene by PCR; then ligating Hla-u, Hla-d and Neor to form an α-HL gene-deleted fragment Hu-Neor-Hd;

2.) cloning the fragment Hu-Neor-Hd into vector pMAD, then performing PCR amplification and restriction enzyme digestion analysis to obtain a gene knockout vector, pMHL-α, which is then subjected to gene modification by transforming it into a Staphylococcus aureus strain RN4220;

3.) Introducing the gene knockout vector pMHL-α which has been subjected to gene modification into a wild type SEs-producing Staphylococcus aureus strain GGMCC0165 by a protoplast transformation method, then culturing and screening the transformed strains to obtain an α-hemolysin-deletion mutant of SEs-producing Staphylococcus aureus. In one preferred embodiment, the α-hemolysin-deletion mutant is constructed by the following steps:

(1). according to the sequence of α-HL gene of a wild type SEs-producing Staphylococcus aureus strain published in Genebank, designing and synthesizing the following two pairs of primers:

| | |
|---|---|
| Hla-uF: | 5′-CGCGGATCCATCGATTACATTT-3′, |
| Hla-uR: | 5′-CGGAATTCTGAGCTGACTATACGTG-3′ |
| Hla-dF: | 5′-CTACTCGAGGTATATGGCAATCAAC-3′ |
| Hla-dR: | 5′-CGCGGATCCCCTCTATAGTGTCATG-3′ |

amplifying the gene fragments Hla-u and Hla-d, which are respectively homologous to the upstream and the downstream of α-HL gene, by PCR, wherein the genomic DNA of a wild type SEs-producing Staphylococcus aureus strain is used as template, and wherein Hla-uF and Hla-uR are used as primers for gene fragment Hla-u, and Hla-dF and Hla-dR are used as primers for gene fragment Hla-d;

(2). according to the sequence of Neor gene published in Genebank, designing and synthesizing the following pair of primers: upstream primer: 5′-GGCGGAATTCATGATTGAACAAGATG-3′ downstream primer: 5′-ATAGCTCGAGATCTCAGAAGAACTCGTCA-3′ amplifying Neor gene by PCR using the above upstream and downstream primers and using pcDNA3.1 as template;

(3). construction of a gene knockout vector pMHL-α: digesting the gene fragments Hla-u and Hla-d and Neor gene obtained in the steps (1) and (2) with suitable restriction enzymes and then ligating the digestion products to form a gene knockout fragment Hu-Neor-Hd, cloning the fragment Hu-Neor-Hd into a shuttle vector, pMAD, then performing transformation, extraction of the desired plasmid, PCR amplification and restriction enzyme digestion analysis, to obtain a gene knockout vector, pMHL-α;

(4). modification of the gene knockout vector pMHL-α: introducing the gene knockout vector pMHL-α into a defective type Staphylococcus aureus strain by electroporation, incubating the obtained strain, extracting the desired DNA from the strain, performing PCR amplification and restriction enzyme digestion analysis to obtain a modified gene knockout vector pMHL-α;

(5). performing gene knockout via the vector pMHL-α: introducing the vector pMHL-α obtained in the step (4) into a wild type SEs-producing Staphylococcus aureus strain by a protoplast transformation method, to obtain an α-hemolysin-deletion mutant of SEs-producing Staphylococcus aureus; Wherein the wild type SEs-producing Staphylococcus aureus strain used in the steps (1) and (5), has been deposited in China General Microbiological Culture Collection Center under accession no. CGMCC0165; the defective type Staphylococcus aureus strain used in the step (4), which can accept any exogenous DNA, has been deposited in American Type Culture Collection Center under accession no. ATCC35556.

The present invention has the following advantages:

1. The present invention provides an α-hemolysin-deletion mutant of SEs-producing Staphylococcus aureus. Through using said mutant, an enterotoxin-containing anti-tumor product with no α-hemolysin can be obtained so that the side effects caused by α-hemolysin can be avoided when the product is used in treatment of tumors.

2. The present invention first apply direct gene knockout technique in a wild type SEs-producing Staphylococcus aureus strain CGMCC0165, thereby providing a more feasible method for constructing an α-hemolysin-deletion mutant of SEs-producing Staphylococcus aureus.

3. The present invention utilize a protoplast transformation method, instead of a phage transduction method usually used in a laboratory, to transform a wild type Staphylococcus aureus strain with an exogenous DNA, so that phage contamination of the resulting genetically engineered strain can be avoided. Therefore, the method of the present invention has high industrial utilization value. Furthermore, the protoplast transformation method has never been used in construction of a genetically engineered strain of Staphylococcus aureus; therefore, the method of the present invention is novel. The method of the present invention can be also used in construction of other genetically engineered strains of Staphylococcus aureus.

### EXAMPLES

### Example 1

### I. PCR Amplification of the Gene Fragments Hla-u and Hla-d which are Homologus to the Upstream and the Downstream of α-Hl Gene of Staphylococcus aureus, Respectively

1). Extraction of Genomic DNA of Staphylococcus aureus

A single colony of Staphylococcus aureus was inoculated on 10 ml of liquid LB medium and incubated overnight with agitation at 37° C. The culture (2 ml) was centrifuged to collect the bacteria. The genomic DNA of Staphylococcus aureus was extracted from the bacteria according to the procedures as described in Current Protocols in Molecular Biology (1995), 3^{rd} edition, p. 39-40, F. M. Ausubel, R. Brent, R. E. Kingston, D. D. Morre, J. G. Seidman, J. A. Smith, K. Struhl; published by John Wiley & Sons, New York City.

Staphylococcus aureus has been deposited in China General Microbiological Culture Collection Center under accession no. CGMCC0165.

2). Primer Design and PCR Condition

The following primers for use in amplification of the gene fragments Hla-u and Hla-d, which are respectively homologous to the upstream and the downstream of α-HL gene, were designed and synthesized.

| | |
|---|---|
| Hla-uF: | 5′-CGCGGATCCATCGATTACATTT-3′, |
| Hla-uR: | 5′-CGGAATTCTGAGCTGACTATACGTG-3′ |
| Hla-dF: | 5′-CTACTCGAGGTATATGGCAATCAAC-3′ |
| Hla-dR: | 5′-CGCGGATCCCCTCTATAGTGTCATG-3′ |

PCR system includes: 10×Pfu buffer 51, dNTP 250 μmol, upstream and downstream primers each 20 pmol, the genomic DNA of Staphylococus aureus CGMCC01650.1 μg, pfu DNA polymerase 2U, and sufficient sterile pure water to make final volume of 50 μl.

### PCR Condition:

the first stage: 95° C. 5 min.

the second stage: 94° C., 45 sec; 45° C., 45 sec; 72° C., 1 min; 5 cycles;

the third stage: 94° C., 45 sec; 55° C., 45 sec; 72° C., 1 min; 25 cycles;

the fourth stage: 72° C., 10 min.

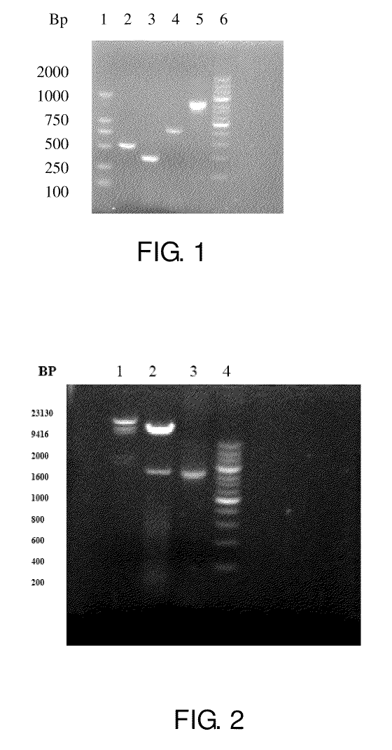

3). recovery of PCR products: The PCR amplification products were separated by electrophoresis on a 1.2% agar gel (FIG. 1). The 354 bp and 500 bp target bands were cut from the agar gel and the PCR products were recovered from the gel by using a gel recovery kit from Takara Biotechnology (Dalian) Co., LTD according to the instruction attached therein.

### II. Ligation of Gene Knockout Fragment

1). PCR amplification of Neor Gene

Upstream primer:
5′-GGCGGAATTCATGATTGAACAAGATG-3′
Downstream primer:
5′-ATAGCTCGAGATCTCAGAAGAACTCGTCA-3′

The bases underlined were restriction site of EcoR I and Xho I, respectively on the upstream and downstream of Neor gene.

PCR system includes: 10×Pfu buffer 5 t, dNTP 250 μmol, upstream and downstream primers each 20 pmol, Neor gene-containing plasmid pcDNA3.1 0.1 μg, pfu DNA polymerase 2U, and sufficient sterile pure water to make final volume of 50 μl.

PCR Condition:

the first stage: 95° C., 5 min.

the second stage: 94° C., 45 sec; 50° C., 45 sec; 72° C., 90 sec; 5 cycles;

the third stage: 94° C., 45 sec; 60° C., 45 sec; 72° C., 90 sec; 25 cycles;

the fourth stage: 72° C., 10 min.

2) Recovery of PCR Products: The products of PCR amplification were separated by electrophoresis on a 1.2% agar gel (FIG. 1). The 798 bp target band was cut from the agar gel and the PCR products were recovered from the gel by using a gel recovery kit from Takara Biotechnology (Dalian) Co., LTD according to the instruction attached therein.

3). Production of Gene Knockout Fragment by Ligation:

The fragments Hla-u, Neor and Hla-d were digested by restriction enzymes BamHI, EcoRI and XhoI, respectively. The digestion products were separated by electrophoresis on a 1.2% agar gel (FIG. 1). The three target bands, respectively containing. Hla-u, Neor and Hla-d, were cut from the agar gel and the digest products were recovered from the gel by using a gel recovery kit from Takara Biotechnology (Dalian) Co., LTD according to the instruction attached therein. The recovered digestion products were ligated to form a gene knockout fragment, Hu-Neor-Hd.

Ligation system: 10×T4 DNA ligase buffer 2.5 μl, Hla-u fragment 4 μl, Hla-d fragment 4 μl, Neor fragment 4 μl, T4 DNA ligase 1 μl, and sufficient dH₂O to make the final volume of 25 μl.

Ligation condition: 16° C., 12 hours

4). PCR Amplification of the Gene Knockout Fragment:

| | |
|---|---|
| upstream primer: | 5′-CGCGGATCCATCGATTACATTT-3′ |
| downstream primer: | 5′-CGCGGATCCCCTCTATAGTGTCATG-3′ |

PCR system includes: 10×Pfu buffer 5 μl, dNTP 250 μmol, upstream and downstream primers each 20 μmol, ligation product Hu-Neor-Hd 5 μl, pfu DNA polymerase 2U, and sufficient sterile pure water to make the final volume of 50 μl.

PCR Condition:

the first stage: 95° C., 5 min.

the second stage: 94° C., 45 sec; 50° C., 45 sec; 72° C., 3 min.; 30 cycles;

the third stage: 72° C., 10 min.

### II. Construction of α-Hemolysin Gene Knockout Vector

The gene knockout fragment Hu-Neor-Hd was digested with a restriction enzyme BamH I and the corresponding digestion products were recovered from the gel by using a gel recovery kit. The digestion products of Hu-Neor-Hd were ligated to a shuttle vector pMAD which had been digested by the same restriction enzyme. The resulting vector was transformed into DH5α competent cells of E. coli. After restriction enzyme digestion analysis and PCR amplification, a correct gene knockout vector pMHL-α was obtained.

### IV. Construction of a Genetically Engineering α-Hemolysin-Deletion Mutant of SEs-Producing Staphylococcus aureus

1). Gene modification of gene knockout vector pMHL-α in Staphylococcus aureus RN4220

The gene knockout vector pMHL-α obtained above was transformed into Staphylococcus aureus RN4220 (a defective type Staphylococcus aureus strain ATCC35556 deposited in American Type Culture Collection Center, which can accept any exogenous gene). A single clone was selected from the culture on TSA (tryptic soy agar) plate containing erythromycin (10 μg/ml). The desired DNA was extracted from the clone, amplified by PCR and subjected to restriction enzyme digestion analysis, then a correct gene knockout vector pMHL-α which had been modified in Staphylococcus aureus RN4220 was selected (FIG. 3).

2). Construction of an Genetically Engineered α-Hemolysin-Deletion Mutant of SEs-Producing Staphylococcus aureus

The modified gene knockout vector pMHL-α was transformed into a wild type Staphylococcus aureus strain CGMCC0165 by protoplast transformation (The protoplast was prepared according to Novick RP. “Genetic systems in Staphylococci” Methods Enzymol, 204:587-636, 1991).

Protoplast transformation experiment: 0.25 ml of plasmid pMHL-α DNA and equal volume of 2×SMM solutions were mixed and 0.5 ml of protoplast was added thereto, then 1.5 ml of 40% PEG 6000 was added immediately and mixed by slight overturn. After 2 minutes, the Staphylococcus aureus strain CGMCC0165 in 5 ml of SMMP culture medium was added and the mixture was centrifuged at 1900 g for 10 minutes to collect the bacteria. The bacteria was incubated with shaking at 32° C. for 2 hours, and then coated onto the DM3 agar plate containing erythromycin (10 μg/ml). The plate was incubated inversely at 32° C. for 2 to 3 days. A single clone was selected and inoculated on 50 ml of TSA culture medium, then incubated with shaking at 30° C. for 2 hours, followed by incubation with shaking at 43° C. for 6 hours. The resulting culture was diluted and coated onto the TSA culture medium containing neomycin (10 μg/ml), then incubated inversely overnight at 42° C. An genetically engineered α-hemolysin-deletion mutant was obtained.

### Example 2

### Molecular identification, Hemolysis Test and Enterotoxin SEC2 Detection for α-Hemolysin-Deletion Mutant of SEs-Producing Staphylococcus aureus

1). Molecular Identification

As most of the encoding sequence (621 bp) of α-HL gene had been replaced by Neor gene (795 bp), PCR amplification was performed by using upstream primer Hla-uF and downstream primer Hla-dR, and using the genomic DNA of the wild type strain CGMCC0165 and the genomic DNA of the α-HL gene-deleted mutant of SEs-producing Staphylococcus aureus as template, respectively, to examine whether the size of PCR products was changed after gene knockout. (FIG. 3)

PCR system includes: 10×Pfu buffer 2.5 μl; dNTP 150-μmol; upstream and downstream primers each 10 pmol; the genomic DNA of Staphylococcus aureus strain CGMCC0165 or the genomic DNA of the α-HL gene-deleted mutant of SEs-producing Staphylococcus aureus as template, 50 ng; pfu DNA polymerase 1U; and sufficient sterile pure water to make the final volume of 25 μl.

PCR Condition:

the first stage: 95° C., 5 min.

the second stage: 94° C., 45 sec; 45° C., 45 sec; 72° C., 2 min.; 5 cycles;

the third stage: 94° C., 45 sec; 55° C., 45 sec; 72° C., 2 min.; 25 cycles;

the fourth stage: 72° C., 10 min.

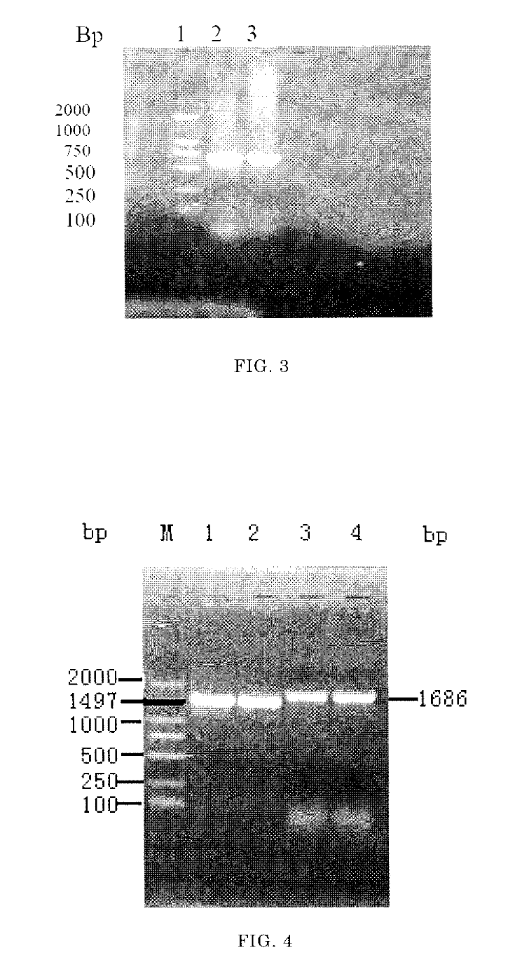

PCR products were separated by electrophoresis on a 1.2% agar gel, the results showed that the PCR products of 1497 bp and 1686 bp in size were obtained by using the genomic DNA of the wild strain CGMCC0165 and the α-HL gene-deleted strain as template, respectively, which met with the theoretic value before and after α-HL gene knockout. This proved that α-hemolysin gene had been successfully replaced with Neor gene, namely, the α-hemolysin-deletion mutant of Staphylococcus aureus had been successfully constructed (FIG. 4).

2). Hemolysis Test

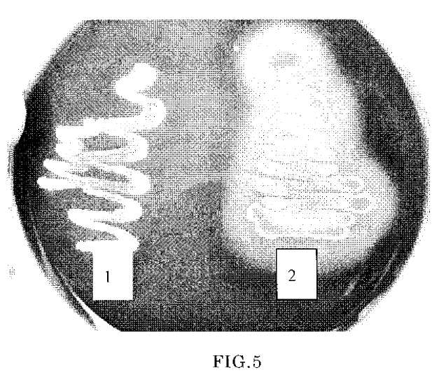

The candidate α-hemolysin-deletion mutant was picked up, and streaked and incubated on a rabbit blood agar plate (containing 5% defibrinized rabbit blood), in the meanwhile, SBE-producing Staphylococcus aureus strain CGMCC0165 before gene knockout was streaked and incubated on the same plate as control. After incubation at 37° C. for more than 16 hours, the plates were observed for hemolysis. The result showed that there was no significant hemolysis circle around the colony of the α-hemolysin-deletion mutant of Staphylococcus aureus, but there are significant hemolysis circle around the colony of the wild type Staphylococcus aureus strain CGMCC0165. This indicated that α-hemolysin-deletion mutant of Staphylococcus aureus had been successfully constructed and α-hemolysin gene had been successfully knocked out (FIG. 5). This mutant, when was fermented to produce a superantigen drug, can avoid the adverse effects caused by α-hemolysin in the fermentation liquor of the wild strain of SEs-producing Staphylococcus aureus; thus the superantigen drug could find use in the clinical anti-tumor therapy.

3). Detection of Enterotoxin C2 (SEC2) Gene by PCR

The following pair of primers were designed and synthesized according to the SEC2 sequence of Staphylococcus aureus published in Genebank:

| | |
|---|---|
| upstream primer: | 5′-GAATTCGAGAGTCAACCAGACCCTA-3′ |
| down primer: | 5′-CTCGAGTTATCCATTCTTTGTTGTA-3′ |

The SEC2 gene of Staphylococcus aureus was amplified by PCR using the genomic DNA of the wild type strain CGMCC0165 and the genomic DNA of α-HL gene-deleted strain as template, respectively, to detect whether SEC2 gene was changed after α-HL gene was knocked out.

PCR system includes: 10×Pfu buffer 2.5 μl, dNTP 150 μmol, upstream and downstream primers each 10 pmol, the genomic DNA of the Staphylococcus aureus strain CGMCC0165 or the genomic DNA of the α-HL gene-deleted mutant of SEs-producing Staphylococcus aureus as template 50 ng, pfu DNA polymerase 1U, and sufficient sterile pure water to make the final volume of 25 μl.

PCR Condition:

the first stage: 95° C., 5 min.

the second stage: 94° C., 45 sec; 45° C., 45 sec; 72° C., 2 min.; 5 cycles;

the third stage: 94° C., 45 sec; 55° C., 45 sec; 72° C., 2 min.; 25 cycles;

the fourth stage: 72° C., 10 min.

The PCR amplification products were analyzed by electrophoresis on 1.2% agar gel. The result showed that the sequences of SEC2 gene before and after deletion of α-hemolysin gene had no difference. This proved that α-hemolysin deletion strain of SBE-producing Staphylococcus aureus was obtained. (FIG. 3)

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An α-hemolysin-deletion mutant of SEs-producing Staphylococcus aureus, which is an genetically engineered stain obtained by deleting α-hemolysin (α-HL) gene of a wild type SEs-producing Staphylococcus aureus strain by a homologuous recombination method.

2. The mutant according to claim 1, wherein the wild type SEs-producing Staphylococcus aureus strain has been deposited in China General Microbiological Culture Collection Center under accession no. CGMCC0165.

3. The mutant according to claim 1, wherein the mutant is prepared by the following steps:
1). amplifying the gene fragment Hla-u which is homologous to the upstream of α-HL gene, the gene fragment Hla-d which is homologous to the downstream of α-HL gene, and neomycin resistance gene (Neor gene) for substituting native α-HL gene by PCR; then ligating Hla-u, Hla-d and Neor to form an α-HL gene-deleted fragment Hu-Neor-Hd;
2). cloning the fragment Hu-Neor-Hd into vector pMAD, then performing PCR amplification and restriction enzyme digestion analysis to obtain a gene knockout vector, pMHL-α, which is then subjected to gene modification by transforming it into a Staphylococcus aureus strain RN4220;
3). introducing the gene knockout vector pMHL-α which has been subjected to gene modification into a wild type SEs-producing Staphylococcus aureus strain GGMCC0165 by a protoplast transformation method, then culturing and screening the transformed strain to obtain an α-hemolysin-deletion mutant of SEs-producing Staphylococcus aureus.

4. A method for constructing an α-hemolysin-deletion mutant of SEs-producing Staphylococcus aureus, comprising the following steps:
(1). according to the sequence of α-HL gene of a wild type SEs-producing Staphylococcus aureus strain published in Genebank, designing and synthesizing the following two pairs of primers:
Hla-uF: 5′-CGCGGATCCATCGATTACATTT-3′,
Hla-uR: 5-CGGAATTCTGAGCTGACTATACGTG-3′
Hla-dF: 5′-CTACTCGAGGTATATGGCAATCAAC-3′
Hla-dR: 5′-CGCGGATCCCCTCTATAGTGTCATG-3′
amplifying the gene fragments Hla-u and Hla-d, which are respectively homologous to the upstream and the downstream of α-HL gene, by PCR, wherein the genomic DNA of a wild type SEs-producing Staphylococcus aureus strain is used as template, and wherein Hla-uF and Hla-uR are used as primers for gene fragment Hla-u, and Hla-dF and Hla-dR are used as primers for gene fragment Hla-d;
(2). according to the sequence of Neor gene published in Genebank, designing and synthesizing the following pair of primers:
upstream primer:
5′-GGCGGAATTCATGATTGAACAAGATG-3′
downstream primer;
5′-ATAGCTCGAGATCTCAGAAGAACTCGTCA-3′
amplifying Neor gene by PCR using the above upstream and downstream primers and using pcDNA3.1 as template;
(3). construction of a gene knockout vector pMHL-α: digesting the gene fragments Hla-u and Hla-d and Neor gene obtained in the steps (1) and (2) with suitable restriction enzymes and then ligating the digestion products to form a gene knockout fragment Hu-Neor-Hd, cloning the fragment Hu-Neor-Hd into a shuttle vector, pMAD, then performing transformation, extraction of the desired plasmid, PCR amplification and restriction enzyme digestion analysis, to obtain a gene knockout vector, pMHL-α;
(4). modification of the gene knockout vector pMHL-α: introducing the gene knockout vector pMHL-α into a defective type Staphylococcus aureus strain by electroporation, incubating the obtained strain, extracting the desired DNA from the strain, performing PCR amplification and restriction enzyme digestion analysis to obtain a modified gene knockout vector pMHL-α;
(5). performing gene knockout via the vector pMHL-α: introducing the vector pMHL-α obtained in the step (4) into a wild type SEs-producing Staphylococcus aureus strain by a protoplast transformation method, to obtain an α-hemolysin-deletion mutant of SEs-producing Staphylococcus aureus.

5. The method according to claim 4, wherein the wild type SEs-producing Staphylococcus aureus strain used in the steps (1) and (5), has been deposited in China General Microbiological Culture Collection Center under accession no. CGMCC0165.


SEQUENCE LISTING (2 sequences)

SEQ ID NO: 1 - DNA, 720 bp, Staphylococcus aureus
gagagtcaaccagaccctacgccagatgagttgcacaaatcaagtgagtttactggtacgatgggtaatatgaaatatttatatgatgatcattatgtatcagcaactaaagttatgtctgtagataaatttttggcacatgatttaatttataacattagtgataaaaaactaaaaaattatgacaaagtgaaaacagagttattaaatgaagatttagcaaagaagtacaaagatgaagtagttgatgtgtatggatcaaattactatgtaaactgctatttttcatccaaagataatgtaggtaaagttacaggtggtaaaacttgtatgtatggaggaataacaaaacatgaaggaaaccactttgataatgggaacttacaaaatgtacttataagagtttatgaaaataaaagaaacacaatttcttttgaagtgcaaactgataagaaaagtgtaacagctcaagaactagacataaaagctaggaattttttaattaataaaaaaaatttgtatgagtttaacagttcaccatatgaaacaggatatataaaatttattgaaaataacggcaatactttttggtatgatatgatgcctgcaccaggcgataagtttgaccaatctaaatatttaatgatgtacaacgacaataaaacggttgattctaaaagtgtgaagatagaagtccaccttacaacaaagaatggataa

SEQ ID NO: 2 - protein, 239 aa, Staphylococcus aureus
ESQPDPTPDELHKSSEFTGTMGNMKYLYDDHYVSATKVMSVDKFLAHDLIYNISDKKLKNYDKVKTELLNEDLAKKYKDEVVDVYGSNYYVNCYFSSKDNVGKVTGGKTCMYGGITKHEGNHFDNGNLGNVLIRVYENKRNTISFEVQTDKKSVTAQELDIKARNFLINKKNLYEFNSSPYETGYIKFIENNGNTFWYDMMPAPGDKFDQSKYLMMYNDNKTVDSKSVKIEVHLTTKNG